(19) United States
(12) Patent Application Publication
(10) Pub. No.: US 2020/0399233 A1
(43) Pub. Date: Dec. 24, 2020

(54) CYCLIC HYDRAZIDES AS FRAGRANCE PRECURSOR COMPOUNDS

(71) Applicant: Henkel AG & Co. KGaA, Duesseldorf (DE)

(72) Inventors: Sascha SCHAEFER, Mettmann (DE); Nadia LEDERMANN, Duisburg (DE)

(21) Appl. No.: 16/903,439

(22) Filed: Jun. 17, 2020

(30) Foreign Application Priority Data
Jun. 21, 2019 (DE) 10 2019 116 765.5

(51) Int. Cl. C07D 263/26; C11B 9/00; A61K 8/49; A61Q 19/00; C07D 413/12; C11D 3/50

(52) U.S. Cl. CPC C07D 263/26; C11B 9/0096; A61K 8/49; C11D 3/50; C07D 413/12; A61K 8/494; A61Q 19/00

(57) ABSTRACT

Fragrance precursor compounds of the formula (I) may be derived from hydrazides and fragrance aldehydes and ketones. The fragrance precursor compounds may be used in composition having washing or cleaning agents, cosmetic agents and air care agents. A method for long-lasting fragrancing of surfaces using the fragrance precursor compounds and agents is also disclosed.

### CROSS-REFERENCE TO RELATED APPLICATIONS

This Patent Application claims priority from German Patent Application No. DE 10 2019 116 765.5 filed on Jun. 21, 2019, the entire disclosure of which is incorporated herein by reference.

### TECHNICAL FIELD

The present disclosure relates to special fragrance precursor compounds of the formula (I) may be derived from hydrazides and fragrance aldehydes and ketones, particularly to washing or cleaning agents, cosmetic agents and air care agents which contain such fragrance precursor compounds. Furthermore, the present disclosure relates to a method for long-lasting fragrancing of surfaces using the fragrance precursor compounds and agents.

### BACKGROUND

Washing or cleaning agents and cosmetic agents generally contain fragrances that impart a pleasant odor to the agents. The fragrances typically mask the odor of the other ingredients, thus giving the user a pleasant impression in terms of odor.

Fragrances, in particular in the field of washing agents, are important components in the composition, since it is intended for both damp laundry and dry laundry to have a fragrance that is pleasant and, where possible, fresh. A fundamental problem associated with the use of fragrances is that they are more or less highly volatile compounds, and yet a long-lasting fragrance effect is desired. In particular in the case of odorants that produce the fresh, light notes of the perfume and are particularly volatile due to their high vapor pressure, it is difficult to achieve the desired long-lasting impression of fragrance.

Fragrances can be released in a delayed manner, for example by so-called fragrance precursor compounds. These fragrance precursor compounds are based on fragrance compounds, in particular fragrance aldehydes or ketones, which are reacted with further compounds which are capable of releasing the actual fragrance compound in a delayed manner by hydrolysis or photochemically. Although both fragrance aldehydes and ketones have been described as starting materials for fragrance precursor compounds, to date there have been no commercially suitable fragrance precursor compounds with ketones. The object was therefore to provide fragrance precursor compounds, in particular based on fragrance ketones, which can be obtained by a simple synthesis. Furthermore, the fragrance precursor compounds should be suitable for use in consumer goods, in particular washing and cleaning agents.

### SUMMARY

The inventors have found that the object can be achieved by the special fragrance precursor compounds of the formula (I), which are derived from hydrazides and fragrance aldehydes and ketones. Surprisingly, it was found that the unwanted splitting off of hydrazine can be avoided by using a cyclic hydrazide.

In one aspect, a fragrance precursor compound may have the formula (I)

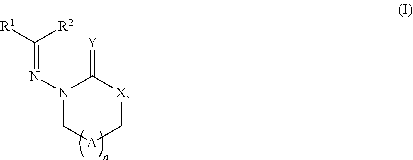

wherein
i) R¹ and R² are selected independently of one another from H, or linear, branched or cyclic, saturated or unsaturated, substituted or unsubstituted hydrocarbon radicals which contain up to 20 carbon atoms and optionally up to 6 heteroatoms;
or
ii) R¹ and R² together with the carbon atom to which they are attached form a cyclic, saturated or unsaturated, hydrocarbon ring with up to 12 carbon atoms, which can contain up to 6 heteroatoms;
   X and Y are selected independently of one another from 0, S, Se or NR³, where R³ is H or a linear or branched, saturated or unsaturated, substituted or unsubstituted hydrocarbon radical with up to 20 carbon atoms, which may optionally contain up to 6 heteroatoms;
   A is —CH₂—, —CHR⁴—, —CR⁴R⁵— or a heteroatom, where R⁴ and R⁵ are selected independently of one another from linear or branched, saturated or unsaturated, substituted or unsubstituted hydrocarbon radicals which contain up to 20 carbon atoms and optionally up to 6 heteroatoms, wherein if several units A are present, these are selected independently of one another,
n=0 to 4,
with the proviso that the radical R¹—C—R² is derived from a fragrance aldehyde or a fragrance ketone of the formula R¹—C(═O)—R², such as a fragrance ketone.

In a second aspect, a washing or cleaning agent may contain at least one fragrance precursor compound.

In a third aspect, an air care agent may contain at least one fragrance precursor compound.

In a fourth aspect, a cosmetic agent may contain at least one fragrance precursor compound.

In a fifth aspect, a method for the preparation of fragrance precursor compounds of the formulas (I) may include reacting a compound of the general formula (IV)

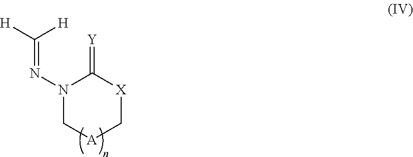

wherein Y, X, A and n are as defined in formula (I), with aldehydes of the formula R¹—C(═O)H, ketones of the formula R¹—C(═O)—R² or mixtures thereof, wherein R¹ and R² are as defined in formula (I).

Finally, in a sixth aspect, a method for long-lasting fragrancing of surfaces, characterized in that a fragrance precursor compound or a washing agent, cleaning agent or cosmetic agent may be applied to the surface to be fragranced, the fragrancing lasts longer than the respective fragrance compound or an identical agent in which the fragrance precursor compound is replaced by the respective fragrance compound is used.

These and other embodiments, features and advantages will become apparent to the person skilled in the art upon studying the following detailed description and claims. Individual described features or embodiments can be combined with other features or embodiments without these having been described in combination. It is understood that the examples contained herein are intended to describe and illustrate the embodiments, but not to limit it, and in particular the embodiments are not limited to the examples.

### DETAILED DESCRIPTION

“At least one,” as used herein, refers to 1 or more, for example 2, 3, 4, 5, 6, 7, 8, 9 or more. In connection with the fragrance precursor compounds described herein, this information does not refer to the absolute amount of molecules, but to the type of the compound. “At least one fragrance precursor compound” therefore means, for example, that only one type of fragrance precursor compound or several different types of fragrance precursor compounds can be contained without providing information about the amount of the individual compounds.

Unless otherwise indicated, all amounts indicated in connection with the method described herein relate to wt. %, based in each case on the total weight of the composition. Furthermore, such quantities which relate to at least one constituent always relate to the total amount of this type of constituent which is contained in the composition, unless explicitly stated otherwise. That means that this type of quantity information, for example in the context of “at least one fragrance precursor compound,” refers to the total quantity of fragrance precursor compounds contained in the composition unless explicitly indicated otherwise.

Numbers without decimals stated herein refer to the full value with one decimal place. For example, “99%” stands for “99.0%.”

Numerical ranges that are specified in the format “in/from x to y” include the stated values. If several numerical ranges are indicated in this format, it is self-evident that all ranges that result from the combination of the various endpoints are also included.

“Substituted” as used herein in connection with the definition of the fragrance precursor compounds of the formulas (I) to (III) means that one hydrogen atom has been replaced by another radical. Suitable radicals include, but are not limited to, linear or branched hydrocarbon groups of up to 22 carbon atoms, such as hydrocarbon groups of up to 10 carbon atoms, including alkyl, alkenyl, alkynyl; —OH, —CN, —NO₂, —C(O)H, —C(O)OR′, —C(O)NR′R″, —NR′—C(O)—R″, —NR′R″, where R′ and R″ are linear or branched alkyl having up to 12 carbon atoms, such as linear or branched alkyl having up to 6 carbon atoms.

The terms “odorant” and “fragrance” are to be used synonymously. An odorant is a compound that has a characteristic odor and helps to achieve a specific fragrance profile of a perfume oil or composition. Odorants also include those compounds which change the fragrance profile of a perfume oil or a composition such that the fragrance is given a certain depth, which the person skilled in the art usually knows as the complexity of a fragrance.

A fragrance precursor compound may include formula (I)

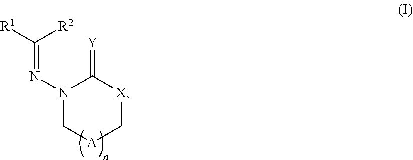

wherein
i) R¹ and R² are selected independently of one another from H, or linear, branched or cyclic, saturated or unsaturated, substituted or unsubstituted hydrocarbon radicals which contain up to 20 carbon atoms and optionally up to 6 heteroatoms;
   or
ii) R¹ and R² together with the carbon atom to which they are attached form a cyclic, saturated or unsaturated, hydrocarbon ring with up to 12 carbon atoms, which can contain up to 6 heteroatoms;
   X and Y are selected independently of one another from 0, S, Se or NR³, where R³ is H or a linear or branched, saturated or unsaturated, substituted or unsubstituted hydrocarbon radical with up to 20 carbon atoms, which may optionally contain up to 6 heteroatoms, from 0 or S;
   A is —CH₂—, —CHR⁴—, —CR⁴R⁵— or a heteroatom, where R⁴ and R⁵ are selected independently of one another from linear or branched, saturated or unsaturated, substituted or unsubstituted hydrocarbon radicals which contain up to 20 carbon atoms and optionally up to 6 heteroatoms, wherein if several units A are present, these are selected independently of one another, such as A is —CH₂—, —CHR⁴— or —CR⁴R⁵—, in particular —CH₂—;
n=0 to 4, such as 0 or 1,
with the proviso that the radical R1-C—R2 is derived from a fragrance aldehyde or a fragrance ketone of the formula R¹—C(═O)—R², such as a fragrance ketone.

In one embodiment, the at least one fragrance precursor compound of the formula (I) is at least one fragrance precursor compound of the formula (II)

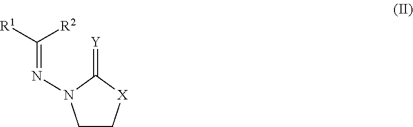

wherein
i) R¹ and R² are selected independently of one another from H, or linear, branched or cyclic, saturated or unsaturated, substituted or unsubstituted hydrocarbon radicals which contain up to 20 carbon atoms and optionally up to 6 heteroatoms;
   or
ii) R¹ and R² together with the carbon atom to which they are attached form a cyclic, saturated or unsaturated, hydrocarbon ring with up to 12 carbon atoms, which can contain up to 6 heteroatoms;
X and Y are selected independently of one another from 0, S, Se or NR³, where R³ is H or a linear or branched, saturated or unsaturated, substituted or unsubstituted hydrocarbon radical with up to 20 carbon atoms, which may optionally contain up to 6 heteroatoms, such as from 0 or S;
with the proviso that the radical R¹—C—R² is derived from a fragrance aldehyde or a fragrance ketone of the formula R¹—C(═O)—R², such as a fragrance ketone.

In a further embodiment, the at least one fragrance precursor compound of the formula (I) is at least one fragrance precursor compound of the formula (III)

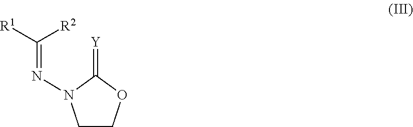

wherein
i) R¹ and R² are selected independently of one another from H, or linear, branched or cyclic, saturated or unsaturated, substituted or unsubstituted hydrocarbon radicals which contain up to 20 carbon atoms and optionally up to 6 heteroatoms;
   or
ii) R¹ and R² together with the carbon atom to which they are attached form a cyclic, saturated or unsaturated, hydrocarbon ring with up to 12 carbon atoms, which can contain up to 6 heteroatoms;
with the proviso that the radical R¹—C—R² is derived from a fragrance aldehyde or a fragrance ketone of the formula R¹—C(═O)—R², such as from a fragrance ketone.

The fragrance can be released from the described fragrance precursor compounds of the formulas (I) to (III), in particular by acid hydrolysis. The fragrance precursor compounds are obtained by reacting a corresponding hydrazide with the fragrance, in this case a fragrance aldehyde or ketone, in particular a fragrance ketone.

In various embodiments, the fragrance aldehyde can be selected from adoxal (2,6,10-trimethyl-9-undecenal), anisaldehyde (4-methoxybenzaldehyde), cymal or cyclamenaldehyde (3-(4-isopropylphenyl)-2-methylpropanal), nympheal (3-(4-isobutyl-2-methylphenyl) propanal), ethyl vanillin, florhydral (3-(3-isopropylphenyl) butanal]), trifernal (3-phenylbutyraldehyde), helional (3-(3,4-methylenedioxyphenyl)-2-methylpropanal), heliotropin, hydroxycitronellal, lauraldehyde, lyral (3- and 4-(4-hydroxy-4-methylpentyl)-3-cyclohexen-1-carboxaldehyde), methylnonylacetaldehyde, filial (3-(4-tert-butylphenyl)-2-methylpropanal), phenylacetaldehyde, undecylenaldehyde, vanillin, 2,6,10-trimethyl-9-undecenal, 3-dodecen-1-al, alpha-n-amylcinnamaldehyde, melonal (2,6-dimethyl-5-heptenal), triplal (2,4-dimethyl-3-cyclohexene-1-carboxaldehyde), 4-methoxybenzaldehyde, benzaldehyde, 3-(4-tert-butylphenyl)propanal, 2-methyl-3-(para-methoxyphenyl)propanal, 2-methyl-4-(2,6,6-timethyl-2(1)-cyclohexen-1-yl)butanal, 3-phenyl-2-propenal, cis-/trans-3,7-dimethyl-2,6-octadien-1-al, 3,7-dimethyl-6-octen-1-al, [(3,7-dimethyl-6-octenyl)oxy]acetaldehyde, 4-isopropylbenzylaldehyde, 1,2,3,4,5,6,7,8-octahydro-8,8-dimethyl-2-naphthaldehyde, 2,4-dimethyl-3-cyclohexene-1-carboxaldehyde, 2-methyl-3-(isopropylphenyl)propanal, 1-decanal, 2,6-dimethyl-5-heptenal, 4-(tricyclo[5.2.1.0(2,6)]-decylidene-8)-butanal, octahydro-4,7-methane-1H-indenecarboxaldehyde, 3-ethoxy-4-hydroxybenzaldehyde, para-ethyl-alpha, alpha-dimethylhydrocinnamaldehyde, alpha-methyl-3,4-(methylenedioxy) hydrocinnamaldehyde, 3,4-methylenedioxybenzaldehyde, alpha-n-hexylcinnamaldehyde, m-cymen-7-carboxaldehyde, alpha-methylphenylacetaldehyde, tetrahydrocitral (3,7-dimethyloctanal), undecenal, 2,4,6-trimethyl-3-cyclohexen-1-carboxaldehyde, 4-(3)(4-methyl-3-pentenyl)-3-cyclohexenecarboxaldehyde, 1-dodecanal, 2,4-dimethylcyclohexen-3-carboxaldehyde, 4-(4-hydroxy-4-methylpentyl)-3-cylohexen-1-carboxaldehyde, 7-methoxy-3,7-dimethyloctan-1-al, 2-methyldecanal, 1-nonanal, 1-octanal, 2,6,10-trimethyl-5,9-undecadienal, 2-methyl-3-(4-tert-butyl)propanal, dihydrocinnamaldehyde, 1-methyl-4-(4-methyl-3-pentenyl)-3-cyclohexen-1-carboxaldehyde, 5- or 6-methoxyhexahydro-4,7-methanindan-1- or -2-carboxaldehyde, 3,7-dimethyloctan-1-al, 1-undecanal, 10-undecen-1-al, 4-hydroxy-3-m ethoxybenzaldehyde, 1-methyl-3-(4-methylpentyl)-3-cyclohexenecarboxaldehyde, 7-hydroxy-3,7-dimethyl-octanal, trans-4-decenal, 2,6-nonadienal, para-tolylacetaldehyde, 4-methylphenylacetaldehyde, 2-methyl-4-(2,6,6-trimethyl-1-cyclohexen-1-yl)-2-butenal, ortho-methoxycinnamaldehyde, 3,5,6-trimethyl-3-cyclohexenecarboxaldehyde, 3,7-dimethyl-2-methylene-6-octenal, phenoxyacetaldehyde, 5,9-dimethyl-4,8-decadienal, peony aldehyde (6,10-dimethyl-3-oxa-5,9-undecadien-1-al), hexahydro-4,7-methanindane-1-carboxaldehyde, 2-methyloctanal, alpha-methyl-4-(1-methylethyl) benzene acetaldehyde, 6,6-dimethyl-2-norpinene-2-propionaldehyde, para-methylphenoxyacetaldehyde, 2-methyl-3-phenyl-2-propen-1-al, 3,5,5-trimethylhexanal, Hexahydro-8,8-dimethyl-2-naphthaldehyde, 3-propylbicyclo[2.2.1]-hept-5-en-2-carbaldehyde, 9-decenal, 3-methyl-5-phenyl-1-pentanal, floral (4,8-Dimethyl-4,9-decadienal), aldehyde C12MNA (2-methylundecanal), liminal (beta-4-dimethylcyclohex-3-ene-1-propan-1-al), methylnonylacetaldehyde, hexanal, trans-2-hexenal and mixtures thereof.

Suitable ketones include, but are not limited to, methyl beta-naphthyl ketone, musk indanone (1,2,3,5,6,7-hexahydro-1,1,2,3,3-pentamethyl-4H-inden-4-on), calone (methylbenzodioxepinone), tonalide (6-acetyl-1,1,2,4,4,7-hexamethyltetralin), alpha-damascone, beta-damascone, delta-damascone, iso-damascone, damascenone, methyldihydrojasmonate (hedione), menthone, carvone, camphor, koavone (3,4,5,6,6-pentamethylhept-3-en-2-one), fenchone, alpha-ionone, beta-ionone, dihydro-beta-ionone, gamma-methyl-ionone, fleuramone (2-heptylcyclopentanone), frambinone methyl ether (4-(4-methoxyphenyl)butan-2-one), dihydrojasmone, cis-jasmone, 1-(1,2,3,4,5,6,7,8-octahydro-2,3,8,8-tetramethyl-2-naphthalenyl)-ethan-1-one and isomers thereof, methylcedrenyl ketone, acetophenone, methylacetophenone, para-m ethoxyacetophenone, methyl-beta-naphthyl ketone, benzylacetone, benzophenone, para-hydroxyphenylbutanone, celery ketone(3-methyl-5-propyl-2-cyclohexenone), 6-isopropyldeca-hydro-2-naphthone, dimethyloctenone, freskomenthe (2-butan-2-ylcyclohexan-1-one), 4-(1-ethoxyvinyl)-3,3,5,5-tetramethylcyclohexanone, methylheptenone, 2-(2-(4-methyl-3-cyclohexen-1-yl) propyl)cyclopentanone, 1-(p-menthen-6(2)yl)-1-propanone, 4-(4-hydroxy-3-methoxyphenyl)-2-butanone, 2-acetyl-3,3-dimethylnorbornane, 6,7-dihydro-1,1,2,3,3-pentamethyl-4 (5H) indanone, 4-damascol, dulcinyl (4-(1,3-benzodioxol-5-yl)butan-2-one), hexalone (1-(2,6,6-trimethyl-2-cyclohexene-1-yl)-1,6-heptadien-3-one), isocyclemone E (2-acetonaphthon-1,2,3,4,5,6,7,8-octahydro-2,3,8,8-tetramethyl), methyl nonyl ketone, methyl cyclocitrone, methyl lavender ketone, orivone (4-tert-amylcyclohexanone), 4-tert-butyl cyclohexanone, delphone (2-pentyl cyclopentanone), muscone (CAS 541-91-3), neobutenone (1-(5,5-dimethyl-1-cyclo-hexenyl)pent-4-en-1-one), plicatone (CAS 41724-19-0), veloutone (2,2,5-trimethyl-5-pentylcyclopentan-1-one), 2,4,4,7-tetramethyl-oct-6-en-3-one, tetramerane (6,10-dimethylundecen-2-one) and mixtures thereof.

The fragrance aldehyde or ketone is selected from dihydro-beta-ionone, frambinone methyl ether, hedione, benzylacetone, calone, decanal, cyclamenaldehyde, melonal and anisaldehyde, in particular from dihydroionone, frambinone methyl ether, hedion, benzylacetone, calone, decanal, cyclamenaldehyde, melonal. In one embodiment, anisaldehyde is not included.

In addition, all customary fragrance aldehydes and/or fragrance ketones which, after reaction, come under the definition of the formula (I) mentioned above, can be used as fragrance aldehydes and/or fragrance ketones, in particular to bring about a pleasant odor sensation in humans. Such fragrance aldehydes and/or fragrance ketones are known to the person skilled in the art and are also described in the patent literature, for example in US 2003/0158079 A1, paragraphs [0154] and For other suitable fragrances, see Steffen Arctander, Aroma Chemicals Volume 1 and Volume 2 (published 1960 and 1969, new edition 2000; ISBN: 0-931710-37-5 and 0-931710-38-3).

To prepare the fragrance precursor compound, a compound of the general formula (IV)

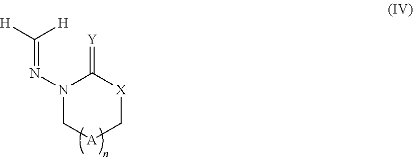

where Y, X, A and n are as defined in formula (I), are reacted with aldehydes, ketones or mixtures of ketones and aldehydes which, after reaction with the carbon atom to which they are attached, have the radical R¹—C—R² as defined in formula (I). Consequently, these are aldehydes of the formula R¹—C(═O)H and ketones of the formula R¹—C(═O)—R², where R¹ and R² are as defined in formula (I).

The reaction takes place under a nitrogen atmosphere. Furthermore, the reaction is carried out in a suitable solvent. Suitable solvents are, for example, aromatic hydrocarbons such as toluene. The reaction is carried out at a temperature in the range from 80 to 150° C., particularly 100 to 140° C. For example, the hydrazide with the general formula shown above is introduced under a nitrogen atmosphere together with the desired ketone and/or aldehyde in the solvent. The reaction mixture is then heated. Frequently, the water separator is then heated under reflux. The reaction product obtained is isolated by conventional methods and optionally purified.

The fragrance precursor compounds of the formulas (I) to (III) described above can be used in the agents of the method as mixtures with at least one further fragrance or at least one further fragrance precursor compound which is different from the fragrance precursor compound according to formula (I).

The other fragrances which may optionally be contained in the compositions are not subject to any particular restrictions. Thus, individual fragrance compounds of natural or synthetic origin, for example of the ester, ether, aldehyde, ketone, alcohol and hydrocarbon type, can be used. Fragrance compounds of the ester type are, for example, benzyl acetate, phenoxyethyl isobutyrate, p-tert-butylcyclohexyl acetate, linalyl acetate, dimethylbenzylcarbinyl acetate (DMBCA), phenylethyl acetate, benzyl acetate, ethylmethylphenyl glycinate, allylcyclohexyl propionate, styrallyl propionate, benzyl salicylate, cyclohexyl salicylate, Floramat, Melusate, and Jasmacyclat. The ethers include, for example, benzyl ethyl ether and ambroxan, the aldehydes include the ones mentioned above, for example the linear alkanals having 8 to 18 carbon atoms, citral, citronellal, citronellyloxyacetaldehyde, cyclamenaldehyde (3-(4-propan-2-ylphenyl)butanal), lilial and bourgeonal, the ketones include, for example, the ionones, [alpha]-isomethylionone and methylcedrylketone, the alcohols include anethole, citronellol, eugenol, geraniol, linalool, phenylethyl alcohol and terpineol, the hydrocarbons mainly include terpenes such as limonene and pinene. In a non-limiting embodiment, mixtures of different fragrances are used, which together produce an appealing fragrance note.

The compositions can also contain natural fragrance mixtures, such as those obtainable from plant sources, for example pine, citrus, jasmine, patchouli, rose or ylang-ylang oil. Likewise suitable are muscatel sage oil, chamomile oil, clove oil, lemon balm oil, mint oil, cinnamon leaf oil, lime blossom oil, juniper berry oil, vetiver oil, olibanum oil, galbanum oil, and labdanum oil, as well as orange blossom oil, neroli oil, orange peel oil, and sandalwood oil. Further conventional fragrances which can be contained in the agents in the context are, for example, the essential oils such as angelica root oil, anise oil, arnica flower oil, basil oil, bay oil, champak flower oil, noble fir oil, noble fir cone oil, elemi oil, eucalyptus oil, fennel oil, spruce needle oil, galbanum oil, geranium oil, ginger grass oil, guaiacum wood oil, gurjun balsam oil, helichrysum oil, ho oil, ginger oil, iris oil, cajeput oil, calamus oil, chamomile oil, camphor oil, canaga oil, cardamom oil, cassia oil, pine needle oil, copaiva balsam oil, coriander oil, spearmint oil, caraway oil, cumin oil, lavender oil, lemon grass oil, kale oil, lime oil, mandarin oil, lemon balm oil, musk seed oil, myrrh oil, clove oil, neroli oil, niaouli oil, olibanum oil, oregano oil, palmarosa oil, patchouli oil, Peru balsam oil, petitgrain oil, pepper oil, peppermint oil, allspice oil, pine oil, rose oil, rosemary oil, sandalwood oil, celery oil, spike lavender oil, star anise oil, turpentine oil, thuja oil, thyme oil, verbena oil, vetiver oil, juniper berry oil, wormwood oil, wintergreen oil, ylang-ylang oil, hyssop oil, cinnamon oil, cinnamon leaf oil, citronella oil, lemon oil and cypress oil as well as ambrettolide, ambroxan, α-amyl cinnammaldehyde, anethole, anisaldehyde, anise alcohol, anisole, anthranilic acid methyl ester, acetophenone, benzylacetone, benzaldehyde, benzoic acid ethyl ester, benzophenone, benzyl alcohol, benzyl acetate, benzyl benzoate, benzyl formate, benzyl valerianate, borneol, bornylacetate, boisambrene forte, α-bromostyrene, n-decylaldehyde, n-dodecyl aldehyde, eugenol, eugenol methyl ether, eucalyptol, farnesol, fenchone, fenchyl acetate, geranyl acetate, geranyl formate, heliotropin, heptin carboxylic acid methyl ester, heptaldehyde, hydroquinone dimethyl ether, hydroxycinnamaldehyde, hydroxycinnamyl alcohol, indole, irone, isoeugenol, isoeugenol methyl ether, isosafrol, jasmone, camphor, carvactol, carvone, p-cresol methyl ether, coumarin, p-methoxyacetophenone, methyl n-amyl ketone, methylanthranilic acid methyl ester, p-methylacetophenone, methylchavicol, p-methylquinoline, methyl-p naphthyl ketone, methyl-n-nonyl acetaldehyde, methyl-n-nonyl ketone, muscone, β-naphthol ethyl ether, β-naphthol methyl ether, nerol, n-nonyl aldehyde, nonyl alcohol, n-octyl aldehyde, p-oxy-acetophenone, pentadecanolide, β-phenylethyl alcohol, phenylacetic acid, pulegone, safrole, salicylic acid isoamyl ester, salicylic acid methyl ester, salicylic acid hexyl ester, salicylic acid cyclohexyl ester, santalol, sandelice, skatole, terpineol, thymene, thymol, troenan, γ-undecalactone, vanillin, veratraldehyde, cinnamaldehyde, cinnamyl alcohol, cinnamic acid, cinnamic acid ethyl ester, cinnamic acid benzyl ester, diphenyl oxide, limonene, linalool, linalyl acetate and propionate, melusate, menthol, menthone, methyl-n-heptenone, pinene, phenylacetaldehyde, terpinyl acetate, citral, cintronellal and mixtures thereof.

It is also possible for the at least one fragrance precursor compounds of the formula (I) to formula (III) to be used with the corresponding aldehydes and/or ketones. According to an embodiment of the method, such compositions are characterized in that the molar ratio of fragrance aldehyde and/or fragrance ketone to the corresponding fragrance precursor compound of the formula (I) to (III) is 20:1 to 1:20, such as 10:1 to 1:10, such as 5:1 to 1:5, such as 3:1 to 1:3, such as 2:1 to 1:2 or 1.2:1 to 1:1.2.

Non-limiting embodiments relate to washing, cleaning, air care or cosmetic agents which contain at least one of the fragrance precursor compounds. In embodiments, the at least one fragrance precursor compound is included in a total amount of 0.001 to 5 wt. %, advantageously 0.005 to 4 wt. %, further advantageously 0.01 to 2 wt. %, in each case based on the total weight of the particular agent.

The washing and cleaning agents to be used in the method can also contain anionic, nonionic, cationic, amphoteric or zwitterionic surfactants or mixtures thereof. Furthermore, these agents can be present in solid or liquid form.

Suitable nonionic surfactants are in particular ethoxylation and/or propoxylation products of alkyl glycosides and/or linear or branched alcohols each having from 12 to 18 C atoms in the alkyl portion and from 3 to 20, such as from 4 to 10, alkyl ether groups. It is possible to use corresponding ethoxylation and/or propoxylation products of N-alkylamines, vicinal diols, fatty acid esters and fatty acid amides, which correspond to the said long-chain alcohol derivatives with regard to the alkyl part, and also of alkyl phenols having 5 to 12 carbon atoms in the alkyl radical.

Suitable anionic surfactants are in particular soaps and those containing sulfate or sulfonate groups having alkali ions as cations. Usable soaps are the alkali salts of saturated or unsaturated fatty acids having from 12 to 18 C atoms. Fatty acids of this kind may also be used in a not completely neutralized form. Usable sulfate-type surfactants include the salts of sulfuric acid semiesters of fatty alcohols having from 12 to 18 C atoms and the sulfation products of the stated nonionic surfactants having a low degree of ethoxylation. Usable sulfonate-type surfactants include linear alkylbenzenesulfonates having from 9 to 14 C atoms in the alkyl portion, alkanesulfonates having from 12 to 18 C atoms, and olefin sulfonates having from 12 to 18 C atoms, resulting from the reaction of corresponding monoolefins with sulfur trioxide, and alpha-sulfo fatty acid esters, resulting from the sulfonation of fatty acid methyl or ethyl esters.

Cationic surfactants are selected from among esterquats and/or quaternary ammonium compounds (QACs) according to general formula (R^{I})(R^{II})(R^{III})(R^{IV})N⁺X⁻, in which R^{I} to R^{IV} represent C₁₋₂₂ alkyl functional groups, C₇₋₂₈ arylalkyl functional groups or heterocyclic functional groups that are the same or different, where two functional groups, or, in the case of aromatic bonding such as in pyridine, even three functional groups form, together with the nitrogen atom, the heterocycle, for example a pyridinium or imidazolinium compound, and X⁻ represents halide ions, sulfate ions, hydroxide ions, or similar anions. QACs may be prepared by reacting tertiary amines with alkalizing agents, for example methyl chloride, benzyl chloride, dimethyl sulfate, dodecyl bromide, but also ethylene oxide. The alkylation of tertiary amines with a long alkyl functional group and two methyl groups is particularly simple; the quaternization of tertiary amines with two long functional groups and a methyl group may also be carried out under mild conditions using methyl chloride. Amines having three long alkyl functional groups or hydroxy-substituted alkyl functional groups are less reactive, and are quaternized using dimethyl sulfate, for example. Examples of suitable QACs are benzalkonium chloride (N-alkyl-N,N-dimethylbenzylammonium chloride), Benzalkon B (m,p-dichlorobenzyldimethyl-C₁₂ alkylammonium chloride, benzoxonium chloride (benzyldodecyl-bis-(2-hydroxyethyl) ammonium chloride), cetrimonium bromide (N-hexadecyl-N, N-trimethylammonium bromide), benzethonium chloride (N, N-dimethyl-N-[2-[2-[p-(1,1,3,3-tetramethylbutyl)phenoxy]ethoxy]ethyl]benzylammonium chloride), dialkyldimethylammonium chlorides such as di-n-decyldimethyl ammonium chloride, didecyldimethyl ammonium bromide, dioctyldimethyl ammonium chloride, 1-cetylpyridinium chloride, and thiazoline iodide, and mixtures thereof. Non-limiting QACs are benzalkonium chlorides having C₈-C₂₂ alkyl functional groups, in particular C₁₂-C₁₄ alkylbenzyldimethyl ammonium chloride.

Non-limiting esterquats are methyl-N-(2-hydroxyethyl)-N, N-di(talgacyloxyethyl) ammonium methosulfate, bis-(palmitoyl)ethylhydroxyethylmethyl ammonium methosulfate or methyl-N,N-bis(acyloxyethyl)-N-(2-hydroxyethyl) ammonium methosulfate. Commercially available examples are the methylhydroxyalkyldialkoyloxyalkyl ammonium methosulfates marketed by Stepan under the trademark Stepantex®, the products from BASF SE known under the trade name Dehyquart®, or the products from the manufacturer Evonik known under the name Rewoquat®.

Furthermore, the washing and cleaning agents may additionally contain other ingredients which further improve the practical and/or aesthetic properties of the composition, depending on the intended use. In the context, they may contain builders, bleaching agents, bleach activators, bleach catalysts, esterquats, silicone oils, emulsifiers, thickeners, electrolytes, pH adjusters, fluorescing agents, dyes, hydrotropes, suds suppressors, anti-redeposition agents, solvents, enzymes, optical brighteners, graying inhibitors, anti-shrink agents, crease-preventing agents, dye transfer inhibitors, color-protection agents, wetting promoters, antimicrobial active ingredients, germicides, fungicides, antioxidants, corrosion inhibitors, clear rinsers, preservatives, antistatic agents, ironing aids, waterproofing and impregnating agents, pearlescing agents, polymers, swelling and anti-slip agents and UV absorbers, without being limited to these.

The amounts of the individual ingredients in the washing and cleaning agents in each case depend on the intended purpose of the composition in question, and a person skilled in the art is in principle familiar with the ranges of the amounts of ingredients that should be used, or can obtain these from the relevant technical literature. Depending on the intended purpose of the composition, the surfactant content, for example, is selected to be higher or lower. For example, the surfactant content of washing agents, for example, can usually be from 10 to 50 wt. %, such as from 12.5 to 30 wt. % or from 15 to 25 wt. %.

The washing and cleaning agents can contain, for example, at least one water-soluble and/or water-insoluble, organic and/or inorganic builder. The water-soluble organic builders include polycarboxylic acids, in particular citric acid and saccharic acids, monomeric and polymeric aminopolycarboxylic acids, in particular methylglycinediacetic acid, nitrilotriacetic acid, ethylenediaminetetraacetic acid and polyaspartic acid, polyphosphonic acids, in particular amino tris(methylenephosphonic acid), ethylenediamine tetrakis(methylenephosphonic acid) and 1-hydroxyethane-1,1-diphosphonic acid, polymeric hydroxy compounds such as dextrin, and polymeric (poly)carboxylic acids, polymeric acrylic acids, methacrylic acids, maleic acids, and mixed polymers thereof, which may also contain, in the polymer, small portions of polymerizable substances, without a carboxylic acid functionality. Compounds of this class which are suitable are copolymers of acrylic acid or methacrylic acid with vinyl ethers, such as vinyl methyl ethers, vinyl esters, ethylene, propylene and styrene, in which the proportion of the acid is at least 50 wt. %. The organic builder substances, in particular for preparing liquid washing and cleaning agents, may be used in the form of aqueous solutions, such as in the form of 30 to 50 wt. % aqueous solutions. All mentioned acids are generally used in the form of the water-soluble salts thereof, in particular alkali salts thereof.

Organic builders, if desired, can be contained in amounts of up to 40 wt. %, in particular up to 25 wt. %, or from 1 wt. % to 8 wt. %. Amounts close to the stated upper limit are used in paste-form or liquid, in particular water-containing, agents. Laundry post-treatment agents, such as softeners, may optionally also be free of organic builders.

In particular alkali silicates and polyphosphates, such as sodium triphosphate, are suitable as water-soluble inorganic builder materials. In particular crystalline or amorphous alkali aluminosilicates, if desired, can be used as water-insoluble, water-dispersible inorganic builder materials in amounts of up to 50 wt. %, such as no greater than 40 wt. %, and in liquid compositions in particular in amounts of from 1 wt. % to 5 wt. %. Among these, crystalline sodium aluminosilicates of washing agent quality, in particular zeolite A, P and optionally X, are optional examples. Amounts close to the stated upper limit are used in solid particulate agents. Suitable aluminosilicates have in particular no particles having a particle size greater than 30 μm and consist up to at least 80 wt. % of particles having a size smaller than 10 μm.

Suitable substitutes or partial substitutes for the stated aluminosilicate are crystalline alkali silicates, which may be present alone or in a mixture with amorphous silicates. The alkali silicates that can be used in the washing or cleaning agents as builders have a molar ratio of alkali oxide to SiO₂ of less than 0.95, in particular from 1:1.1 to 1:12, and may be present in amorphous or crystalline form. Non-limiting alkali silicates are sodium silicates, in particular amorphous sodium silicates, having a Na₂O:SiO₂ molar ratio of from 1:2 to 1:2.8. Crystalline phyllosilicates of the general formula Na₂SiₓO₂ₓ₊₁.H₂O, where x, referred to as the module, is a number from 1.9 to 4, y is a number from 0 to 20, and non-limiting values for x are 2, 3 or 4, are optionally used as crystalline silicates, which may be present alone or in a mixture with amorphous silicates. Non-limiting crystalline phyllosilicates are those in which x in the stated general formula assumes the values 2 or 3. In particular, both beta- and delta-sodium disilicates (Na₂Si₂O₅.yH₂O) are optional examples. Practically water-free crystalline alkali silicates of the above general formula, in which x is a number from 1.9 to 2.1, which alkali silicates are produced from amorphous alkali silicates, may also be used. In a further embodiment, a crystalline sodium phyllosilicate having a module of from 2 to 3, as can be produced from sand and soda, is used. Crystalline sodium silicates having a module in the range of from 1.9 to 3.5 are used in a further embodiment of the textile treatment or cleaning agents. If alkali aluminosilicate, in particular zeolite, is also present as an additional builder, the weight ratio of aluminosilicate to silicate, based in each case on water-free active substances, ranges from 1:10 to 10:1. In compositions containing both amorphous and crystalline alkali silicates, the weight ratio of amorphous alkali silicate to crystalline alkali silicate ranges from 1:2 to 2:1 and in particular 1:1 to 2:1.

Builder substances are, if desired, contained in amounts of up to 60 wt. %, in particular from 5 wt. % to 40 wt. %. Laundry post-treatment agents, for example softeners, are free of inorganic builders.

Suitable ingredients and framework compositions for washing and cleaning agent compositions (for example for detergents and fabric softeners) are disclosed, for example, in EP 3 110 393 B1.

Suitable ingredients and framework compositions for cosmetic agents, such as hair care agent compositions, are disclosed, for example, in DE 102017215071 A1.

### Examples

### Production Example 1: 3-amino-1,3-oxazolidin-2-one (A)

A 250-mL Schlenk flask was secured and heated. 1.74 g of 2-oxazolidinone in 50 mL of dry 1,4-dioxane were introduced under a nitrogen atmosphere. 0.96 g of sodium hydride was added at room temperature. The mixture was reacted at 60° C. for 1 h. The mixture was then cooled to room temperature. Cooled by an ice bath, 2.38 g of hydroxylamine-O-sulfonic acid were then added in portions. The ice bath was removed and the reaction mixture was stirred at room temperature for 24 h. The mixture was cooled by an ice bath and ethanol and distilled water were added. The crude product was filtered through Celite and the solvent of the filtrate was removed under reduced pressure. The product was dissolved in water, the solution was covered with methanol and stored in the refrigerator. The desired product A was obtained in a mixture with unreacted 2-oxazolidinone (m=1.50 g).

### Example 2: 3-{[4-(2,6,6-trimethylcyclohex-2-en-1-yl)butan-2-ylidene]amino}-1,3-oxazolidin-2-one (B)

0.60 g of 3-amino-1,3-oxazolidin-2-one in 50 mL of toluene were placed in a 250-mL one-necked flask. 2.50 g of dihydro-beta-ionone and 0.04 g of p-toluenesulfonic acid monohydrate were added and the mixture was stirred at 125° C. until conversion was complete (3 h). The mixture was then cooled to room temperature, the solvent was removed under reduced pressure and purified by column chromatography (mobile phase:hexane:ethyl acetate). The desired product B was obtained as a yellowish oil (m=0.85 g, 48% over two stages). R_{f}=0.49 (hexane:ethyl acetate; 2:1). FTIR (film): \tilde{v}=2926, 1755, 1638, 1477, 1382, 1208, 1033, 755 cm⁻¹. ¹H-NMR (400 MHz, DMSO-d₆): δ=4.35 (t, J=7.7 Hz, 2H), 3.75 (t, J=7.7 Hz, 2H), 2.40-2.45 (m, 2H), 2.18-2.21 (m, 2H), 2.00-2.02 (m, 1H), 1.95 (s, 3H), 1.85-1.89 (m, 1H), 1.58 (s, 3H), 1.50-1.52 (m, 2H), 1.35-1.41 (m, 2H), 1.00 (s, 6H) ppm. ¹³C-NMR (125.8 MHz, DMSO-d₆): δ=174.7 (C_{quart}), 155.4 (C_{quart}), 136.3 (C_{quart}), 127.6 (C_{quart}), 62.1 (CH₂), 48.5 (CH₂), 39.1 (CH₂), 35.0 (CH), 32.6 (CH₂), 29.9 (2 CH₃), 28.8 (CH₂), 28.5 (CH), 24.8 (CH₃), 21.1 (CH₂), 19.4 (CH₃) ppm. MS (ESI, positive ion): 279 ([M+H]⁺, 100%).

### Example 3: 3-{[4-(4-methoxyphenyl)butan-2-ylidene]amino}-1,3-oxazolidin-2-one (C)

0.70 g of 3-amino-1,3-oxazolidin-2-one in 50 mL of toluene were placed in a 250-mL one-necked flask. 2.70 g of frambinone methyl ether and 0.04 g of p-toluenesulfonic acid monohydrate were added and the mixture was stirred at 125° C. until conversion was complete (2 h). The mixture was then cooled to room temperature, the solvent was removed under reduced pressure and purified by column chromatography (mobile phase:hexane:ethyl acetate). The desired product (C) was obtained as a yellowish oil (m=0.85 g, 64% over two stages). R_{f}=0.22 (hexane:ethyl acetate; 2:1). FTIR (film): \tilde{v}=2912, 1752, 1611, 1511, 1383, 1241, 1031, 824, 754, 707 cm⁻¹. ¹H-NMR (400 MHz, DMSO-d₆): δ=7.18-7.21 (m, 2H), 6.86-7.90 (m, 2H), 4.35 (t, J=7.8 Hz, 2H), 4.05 (t, J=5.5 Hz, 2H), 3.75 (s, 3H), 2.76-2.85 (m, 2H), 2.55-2.61 (m, 2H), 1.95 (s, 3H) ppm. ¹³C-NMR (125.8 MHz, DMSO-d₆): δ=174.3 (C_{quart}), 157.9 (C_{quart}), 155.5 (C_{quart}), 133.3 (C_{quart}), 129.5 (2 CH), 114.0 (2 CH), 62.1 (CH₂), 55.3 (CH₃), 48.5 (CH₂), 40.5 (CH₂), 31.3 (CH₂), 19.2 (CH₃) ppm. MS (ESI, positive ion): 263([M+H]⁺, 100%).

### Example 4: Methyl-{(3-[(2-oxo-1,3-oxazolidin-3-yl)imino]-2-pentylcyclopentyl}acetate (D)

0.60 g of 3-amino-1,3-oxazolidin-2-one in 50 mL of toluene were placed in a 250-mL one-necked flask. 2.95 g of hedione and 0.04 g of p-toluenesulfonic acid monohydrate were added and the mixture was stirred at 125° C. until conversion was complete (3 h). The mixture was then cooled to room temperature, the solvent was removed under reduced pressure and purified by column chromatography (mobile phase:hexane:ethyl acetate). The desired product (D) was obtained as a yellowish oil (m=0.52 g, 34% over two stages). R_{f}=0.14 (hexane:ethyl acetate; 2:1). FTIR (film): \tilde{v}=2930, 1734, 1384, 1188, 1033, 753, 700 cm⁻¹. ¹H-NMR (400 MHz, DMSO-d₆): δ=4.30-4.38 (m, 2H), 3.70-3.79 (m, 2H), 3.60 (s, 3H), 2.49-2.52 (m, 2H), 2.30-2.37 (m, 2H), 2.10-2.18 (m, 1H), 1.95-2.02 (m, 1H), 1.45-1.55 (m, 2H), 1.15-1.35 (m, 8H), 0.85 (t, J=7.2 Hz, 3H) ppm. ¹³C-NMR (125.8 MHz, DMSO-d₆): δ=182.5 (C_{quart}), 172.7 (C_{quart}), 155.2 (C_{quart}), 62.0 (CH₂), 51.7 (CH₃), 49.6 (CH), 48.0 (CH₂), 38.1 (CH), 37.0 (CH₂), 33.3 (CH₂), 31.9 (CH₂), 31.7 (CH₂), 28.5 (CH₂), 26.1 (CH₂), 22.3 (CH₂), 14.2 (CH₃) ppm. MS (ESI, positive ion): 311 ([M+H]⁺, 100%).

### Example 5: 3-{[4-phenylbutan-2-ylidene]amino}-1,3-oxazolidin-2-one (E)

0.60 g of 3-amino-1,3-oxazolidin-2-one in 50 mL of toluene were placed in a 250-mL one-necked flask. 2.00 g of benzylacetone and 0.04 g of p-toluenesulfonic acid monohydrate were added and the mixture was stirred at 125° C. until conversion was complete (3 h). The mixture was then cooled to room temperature, the solvent was removed under reduced pressure and purified by column chromatography (mobile phase:hexane:ethyl acetate). The desired product (E) was obtained as a yellowish oil (m=0.45 g, 38% over two stages). R_{f}=0.33 (hexane:ethyl acetate; 2:1). ¹H-NMR (400 MHz, DMSO-d₆): δ=7.12-7.21 (m, 5H), 4.38 (t, J=7.6 Hz, 2H), 3.75 (t, J=7.8 Hz, 2H), 2.89-2.92 (m, 2H), 2.65-2.71 (m, 2H), 2.02 (s, 3H) ppm. ¹³C-NMR (125.8 MHz, DMSO-d₆): δ=175.4 (C_{quart}), 155.9 (C_{quart}), 141.4 (C_{quart}), 128.7 (4 CH), 126.5 (CH), 62.2 (CH₂), 49.1 (CH₂), 40.9 (CH₂), 32.9 (CH₂), 17.9 (CH₃) ppm. MS (ESI, positive ion): 233 ([M+H]⁺, 100%).

### Example 6: 3-{[7-methyl-2H-1,5-benzodioxepin-3 (4H)-ylidene] amino}-1,3-oxazolidin-2-one (F)

0.70 g of 3-amino-1,3-oxazolidin-2-one in 50 mL of toluene were placed in a 250-mL one-necked flask. 2.70 g of calone and 0.04 g of p-toluenesulfonic acid monohydrate were added and the mixture was stirred at 125° C. until conversion was complete (3 h). The mixture was then cooled to room temperature, the solvent was removed under reduced pressure and purified by column chromatography (mobile phase:hexane:ethyl acetate). The desired product (F) was obtained as a yellowish oil (m=0.33 g, 26% over two stages). R_{f}=0.40 (hexane:ethyl acetate; 2:1). FTIR (film): \tilde{v}=2920, 1749, 1503, 1386, 1184, 1019, 801, 747, 703, 588 cm⁻¹. ¹H-NMR (400 MHz, DMSO-d₆): δ=6.85-6.90 (m, 1H), 6.72-6.78 (m, 1H), 6.68-6.71 (m, 1H), 4.95 (d, J=9.5 Hz, 2H), 4.85 (d, J=5.7 Hz, 2H), 4.45 (t, J=7.5 Hz, 2H), 3.85 (t, J=7.6 Hz, 2H), 2.20 (s, 3H) ppm. ¹³C-NMR (125.8 MHz, DMSO-d₆): δ=167.9 (C_{quart}), 155.2 (C_{quart}), 147.9 (C_{quart}), 134.0 (C_{quart}), 132.5 (C_{quart}), 124.9 (CH), 122.0 (CH), 120.0 (CH), 71.5 (CH₂), 70.6 (CH₂), 62.6 (CH₂), 48.9 (CH₂), 20.5 (CH₃) ppm. MS (ESI, positive ion): 263 ([M+H]⁺, 100%).

### Example 7: 3-[decylideneamino]-1,3-oxazolidin-2-one (G)

0.60 g of 3-amino-1,3-oxazolidin-2-one from Example 1 in 50 mL of toluene were placed in a 250-mL one-necked flask. 2.00 g of decanal and 0.04 g of p-toluenesulfonic acid monohydrate were added and the mixture was stirred at 125° C. until conversion was complete (2 h). The mixture was then cooled to room temperature, the solvent was removed under reduced pressure and purified by column chromatography (mobile phase:hexane:ethyl acetate). The desired product (G) was obtained as a yellowish oil (m=0.57 g, 48%, over two stages). R_{f}=0.45 (hexane:ethyl acetate; 1:1). FTIR (film): \tilde{v}=2923, 1753, 1407, 1224, 1098, 1039, 754 cm⁻¹. ¹H-NMR (400 MHz, DMSO-d₆): δ=7.05 (t, J=5.4 Hz, 1H), 4.40 (t, J=7.7 Hz, 2H), 3.73 (t, J=7.7 Hz, 2H), 2.25 (q, J=7.6 Hz, 2H), 1.15 (bs, 14H), 0.85 (t, J=7.2 Hz, 3H) ppm. ¹³C-NMR (125.8 MHz, DMSO-d₆): δ=154.3 (C_{quart}), 148.0 (CH), 61.7 (CH₂), 42.5 (CH₂), 32.4 (CH₂), 31.7 (CH₂), 29.6 (CH₂), 29.5 (CH₂), 29.3 (CH₂), 29.1 (CH₂), 26.6 (CH₂), 22.5 (CH₂), 14.2 (CH₃) ppm. MS (ESI, positive ion): 241 ([M+H]⁺, 100%).

### Example 8: 3-({(2-methyl-3-[4-(propan-2-yl)phenyl]propylidene} amino)-1,3-oxazolidin-2-one (H)

0.70 g of 3-amino-1,3-oxazolidin-2-one in 50 mL of toluene were placed in a 250-mL one-necked flask. 2.90 g of cyclamenaldehyde and 0.04 g of p-toluenesulfonic acid monohydrate were added and the mixture was stirred at 125° C. until conversion was complete (3 h). The mixture was then cooled to room temperature, the solvent was removed under reduced pressure and purified by column chromatography (mobile phase:hexane:ethyl acetate). The desired product was obtained as a colorless solid (H) (m=0.43 g, 32% over two stages). R_{f}=0.12 (hexane:ethyl acetate; 2:1). FTIR (film): \tilde{v}=2960, 1748, 1412, 1242, 1095, 1040, 754 cm⁻¹. ¹H-NMR (400 MHz, DMSO-d₆): δ=7.10-7.16 (m, 4H), 7.08 (d, J=5.7 Hz, 1H), 4.42 (t, J=7.8 Hz, 2H), 3.68-3.72 (m, 2H), 2.80-2.88 (m, 2H), 2.62-2.72 (m, 1H), 2.55-2.60 (m, 1H), 1.20 (d, J=6.8 Hz, 6H), 1.05 (d, J=6.8 Hz, 3H) ppm. ¹³C-NMR (125.8 MHz, DMSO-d₆): δ=154.3 (CH), 151.6 (C_{quart}), 146.3 (C_{quart}), 137.2 (C_{quart}), 129.2 (2 CH), 126.3 (2 CH), 61.7 (CH₂), 42.2 (CH₂), 39.7 (CH₂), 38.2 (CH), 33.4 (CH), 24.2 (2 CH₃), 17.7 (CH₃) ppm. MS (ESI, positive ion): 275 ([M+H]⁺, 100%).

### Example 9: 3-{[2,6-dimethylhept-5-en-1-ylidene]amino}-1,3-oxazolidin-2-one (I)

0.70 g of 3-amino-1,3-oxazolidin-2-one in 50 mL of toluene were placed in a 250-mL one-necked flask. 2.13 g of melonal and 0.04 g of p-toluenesulfonic acid monohydrate were added and the mixture was stirred at 125° C. until conversion was complete (3 h). The mixture was then cooled to room temperature, the solvent was removed under reduced pressure and purified by column chromatography (mobile phase:hexane:ethyl acetate). The desired product (I) was obtained as a yellowish oil (m=0.36 g, 32% over two stages). R_{f}=0.25 (hexane:ethyl acetate; 2:1). ¹H-NMR (400 MHz, DMSO-d₆): δ=6.95 (d, J=6.0 Hz, 1H), 5.10 (t, J=7.3 Hz, 1H), 4.42 (t, J=7.5 Hz, 2H), 3.73 (t, J=7.0 Hz, 2H), 2.35-2.45 (m, 1H), 1.95-1.98 (m, 2H), 1.65 (s, 3H), 1.58 (s, 3H), 1.49-1.51 (m, 1H), 1.35-1.43 (m, 1H), 1.05 (d, J=6.9 Hz, 3H) ppm. ¹³C-NMR (125.8 MHz, DMSO-d₆): δ=154.4 (C_{quart}), 152.1 (CH), 131.4 (C_{quart}), 124.4 (CH), 61.7 (CH₂), 42.2 (CH₂), 36.3 (CH), 31.1 (CH₂), 25.8 (CH₃), 25.5 (CH₂), 18.1 (CH₃), 17.9 (CH₃) ppm. MS (ESI, positive ion): 225 ([M+H]⁺, 100%).

### Example 10: 3-{[(4-methoxyphenyl)methylidene]amino}-1,3-oxazolidin-2-one (J)

1.50 g of 3-amino-1,3-oxazolidin-2-one from Example 1 in 50 mL of toluene were placed in a 250-mL two-necked flask under a nitrogen atmosphere. 3.00 g of anisaldehyde and 0.06 g of p-toluenesulfonic acid monohydrate were added and the mixture was stirred at 125° C. until conversion was complete (3 h). The mixture was then cooled to room temperature, the solvent was removed under reduced pressure and purified by column chromatography (mobile phase:hexane:ethyl acetate). The desired product (J) was obtained as a colorless solid (m=0.52 g, 48% over two stages). R_{f}=0.12 (hexane:ethyl acetate; 2:1). FTIR (film): \tilde{v}=2917, 1752, 1605, 1471, 1414, 1243, 1090, 1023, 829, 749, 531 cm⁻¹. ¹H-NMR (400 MHz, DMSO-d₆): δ=7.68-7.71 (m, 2H), 7.28-7.31 (m, 2H), 6.95-7.00 (m, 1H), 4.39 (t, J=7.3 Hz, 2H), 3.94 (t, J=7.7 Hz, 2H), 3.45 (s, 3H) ppm. ¹³C-NMR (125.8 MHz, DMSO-d₆): δ=160.9 (C_{quart}), 154.4 (C_{quart}), 143.8 (CH), 128.9 (CH), 127.3 (C_{quart}), 114.7 (CH), 61.9 (CH₂), 55.5 (CH₃), 42.5 (CH₂) ppm. MS (ESI, positive ion): 221 ([M+H]⁺, 100%).

### Example 11: Smell Test

The following values are the average of the ratings of at least three testers. The samples were prepared as follows: An approximately 200-mM solution of the corresponding pure odorants and their precursors in diethyl ether or ethanol was prepared. The solution was then applied to paper test strips by immersing them in the pre-made solution. After the solvent front had risen by about 2-3 cm, the test strip was placed on a laboratory tray to dry at room temperature and smelled directly after the specified time or sprayed with water mist or a pH=3 buffer solution and then smelled. Scale: 0: No smell perceptible; 6: Very strong fragrance impression.

| | | | | 6 h | 6 h | | 24 h | 24 h | | 48 h | 48 h | | |
|---|---|---|---|---|---|---|---|---|---|---|---|---|---|
| Solvent | Compound | 15 min | 6 h | H2O | pH3 | 24 h | H2O | pH3 | 48 h | H2O | pH3 | 72 h | 144 h |
| EtOH | β- | 6.00 | | | | 0.67 | | | 0.34 | | | | |
| | dihydroionone | | | | | | | | | | | | |
| | Dihydro-beta- | 3.75 | | | | 1.17 | | | 0.34 | | | | |
| | ionone | | | | | | | | | | | | |
| | precursor (B) | | | | | | | | | | | | |
| | Frambinone | 2.67 | | 2.17 | | | | | 1.67 | | | | 0.00 |
| | methyl ether | | | | | | | | | | | | |
| | Frambinone | 4.34 | | 2.34 | | | | | 1.83 | | | | 0.50 |
| | methyl ether | | | | | | | | | | | | |
| | precursor (C) | | | | | | | | | | | | |
| | Hedione | 1.00 | 1.00 | 0.83 | 1.34 | 0.67 | 1.00 | 0.50 | 0.67 | 0.50 | 0.83 | 0.34 | 0.00 |
| | Hedione | 3.34 | 2.75 | 2.83 | 2.34 | 3.50 | 2.67 | 2.00 | 2.34 | 3.00 | 2.34 | 1.50 | 1.17 |
| | precursor (D) | | | | | | | | | | | | |
| | Benzylacetone | 5.00 | 1.00 | | | 0.00 | | | 0.75 | | | 0.00 | |
| | Benzylacetone | 3.00 | 4.00 | | | 2.33 | | | 2.75 | | | 1.25 | |
| | Precursor (E) | | | | | | | | | | | | |
| | Calone | 5.00 | | | | | | 3.00 | | 1.75 | | | |
| | Calone- | 1.00 | | | | | | 3.17 | | 1.83 | | | |
| | Precursor (F) | | | | | | | | | | | | |
| | Decanal | 6.00 | | | | 2.00 | | | | | 0.00 | | |
| | Decanal | 4.17 | | | | 2.67 | | | | | 1.50 | | |
| | Precursor (G) | | | | | | | | | | | | |
| | Cyclamenaldehyde | 5.33 | | | 1.33 | | | | | | | | |
| | Cyclamenaldehyde | 2.17 | | | 2.50 | | | | | | | | |
| | Precursor | | | | | | | | | | | | |
| | (H) | | | | | | | | | | | | |
| Et₂O | β- | 5.67 | | | | 1.17 | | | 0.67 | | | 0.17 | .00 |
| | Dihydroionone | | | | | | | | | | | | |
| | Dihydro-beta- | 3.34 | | | | 2.34 | | | 1.00 | | | 0.67 | 0.75 |
| | ionone | | | | | | | | | | | | |
| | precursor (B) | | | | | | | | | | | | |
| | Frambinone | 3.34 | | | 1.50 | | | | | | | | |
| | methyl ether | | | | | | | | | | | | |
| | Frambinone | 2.67 | | | 2.17 | | | | | | | | |
| | methyl ether | | | | | | | | | | | | |
| | precursor (C) | | | | | | | | | | | | |
| | Hedione | 1.67 | | | | 0.67 | 1.50 | 1.50 | 1.00 | 1.67 | 1.67 | 0.50 | 0.00 |
| | Hedione | 4.50 | | | | 3.00 | 2.67 | 2.83 | 1.84 | 1.84 | 2.67 | 1.50 | 1.50 |
| | precursor (D) | | | | | | | | | | | | |
| | Benzylacetone | 4.67 | | | | 0.30 | | | 2.25 | 1.75 | | 0.00 | |
| | Benzylacetone | 3.67 | | | | 3.67 | | | 4.00 | 2.75 | | 3.00 | |
| | Precursor (E) | | | | | | | | | | | | |
| | Calone | 5.00 | | | | | | | | | 1.50 | | |
| | Calone- | 2.67 | | | | | | | | | 2.50 | | |
| | Precursor (F) | | | | | | | | | | | | |
| | Decanal | 6.00 | 1.67 | 1.33 | 1.00 | 2.67 | 0.50 | | 0.25 | 1.50 | 2.25 | 0.00 | |
| | Decanal | 4.00 | 3.17 | 2.33 | 3.33 | 3.67 | 1.67 | | 1.25 | 2.75 | 2.75 | 3.00 | |
| | Precursor (G) | | | | | | | | | | | | |
| | Melonal | 3.67 | | 0.33 | | | | | 0.67 | | | | |
| | Melonal- | 2.33 | | 0.33 | | | | | 1.33 | | | | |
| | Precursor (I) | | | | | | | | | | | | |

1. A fragrance precursor compound comprising the formula (I) 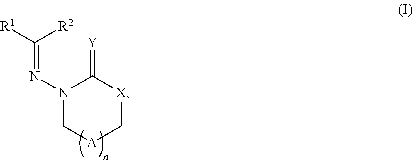
wherein
i) R¹ and R² are selected independently of one another from H, or linear, branched or cyclic, saturated or unsaturated, substituted or unsubstituted hydrocarbon radicals which contain up to 20 carbon atoms and optionally up to 6 heteroatoms;
or
ii) R¹ and R² together with the carbon atom to which they are attached form a cyclic, saturated or unsaturated, hydrocarbon ring with up to 12 carbon atoms and optionally up to 6 heteroatoms;
X and Y are selected independently of one another from 0, S, Se, or NR³, where R³ is H, or a linear or branched, saturated or unsaturated, substituted or unsubstituted hydrocarbon radical with up to 20 carbon atoms and optionally up to 6 heteroatoms;
A is —CH₂—, —CHR⁴—, —CR⁴R⁵— or a heteroatom, where R⁴ and R⁵ are selected independently of one another from linear or branched, saturated or unsaturated, substituted or unsubstituted hydrocarbon radicals which contain up to 20 carbon atoms and optionally up to 6 heteroatoms, wherein if several units A are present, these are selected independently of one another;
n=0 to 4,
wherein the radical R¹—C—R² is derived from a fragrance aldehyde or a fragrance ketone of the formula R¹—C(═O)—R².

2. The fragrance precursor compound according to claim 1, wherein the at least one fragrance precursor compound of the formula (I) is at least one fragrance precursor compound having the formula (II) 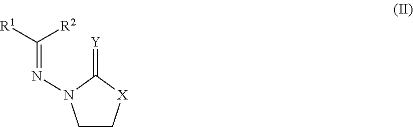
wherein
i) R¹ and R² are selected independently of one another from H, or linear, branched or cyclic, saturated or unsaturated, substituted or unsubstituted hydrocarbon radicals which contain up to 20 carbon atoms and optionally up to 6 heteroatoms;
or
ii) R¹ and R² together with the carbon atom to which they are attached form a cyclic, saturated or unsaturated, hydrocarbon ring with up to 12 carbon atoms and optionally up to 6 heteroatoms;
X and Y are selected independently of one another from 0, S, Se, or NR³, where R³ is H, or a linear or branched, saturated or unsaturated, substituted or unsubstituted hydrocarbon radical with up to 20 carbon atoms and optionally up to 6 heteroatoms;
wherein the radical R¹—C—R² is derived from a fragrance aldehyde or a fragrance ketone of the formula R¹—C(═O)—R².

3. The fragrance precursor compound according to claim 1, wherein the at least one fragrance precursor compound of formula (I) is at least one fragrance precursor compound of formula (III) 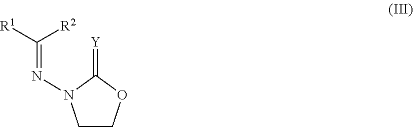
wherein
i) R¹ and R² are selected independently of one another from H, or linear, branched or cyclic, saturated or unsaturated, substituted or unsubstituted hydrocarbon radicals which contain up to 20 carbon atoms and optionally up to 6 heteroatoms;
or
ii) R¹ and R² together with the carbon atom to which they are attached form a cyclic, saturated or unsaturated, hydrocarbon ring with up to 12 carbon atoms and optionally up to 6 heteroatoms;
wherein the radical R¹—C—R² is derived from a fragrance aldehyde or a fragrance ketone of the formula R¹—C(═O)—R².

4. The fragrance precursor compound according to claim 1, wherein the compound is obtained by the reaction of a hydrazide with at least one fragrance aldehyde or ketone selected from the group consisting of dihydro-beta-ionone, frambinone methyl ether, hedione, benzylacetone, calone, decanal, cyclamenaldehyde, melonal, anisaldehyde, or mixtures thereof.

5. A composition comprising:
a washing agent, a cleaning agent, an air care agent, a cosmetic agent, or combinations thereof; and
at least one fragrance precursor compound according to claim 1, wherein the compound is present in a total amount ranging from 0.001 and 5 wt. % in each case based on the total weight of the composition.

6. The composition according to claim 5, further comprising at least one surfactant selected from the group consisting of anionic surfactants, cationic surfactants, nonionic surfactants, zwitterionic surfactants, amphoteric surfactants, or mixtures thereof;
and/or wherein the composition is present in solid or liquid form.

7. A method of preparing fragrance precursor compounds of the formula (I) according to claim 1, wherein the method comprises:
reacting a compound according to the general formula (IV) 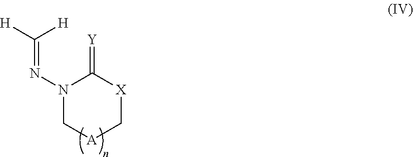
with aldehydes of the formula R¹—C(═O)H, ketones of the formula R¹—C(═O)—R², or mixtures thereof, wherein R¹ and R² are as defined in formula (I), and wherein Y, X, A and n are defined as in formula (I) of claim 1.

8. The fragrance precursor compound according to claim 4, wherein the hydrazide is 3-amino-1,3-oxazolidin-2-one.
